(19) United States
(12) Patent Application Publication
(10) Pub. No.: US 2010/0249783 A1
(43) Pub. Date: Sep. 30, 2010

(54) DRUG-ELUTING IMPLANT COVER

(75) Inventor: Hai H. TRIEU, Cordova, TN (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., Warsaw, IN (US)

(21) Appl. No.: 12/409,899

(22) Filed: Mar. 24, 2009

(51) Int. Cl. A61B 17/58

(52) U.S. Cl. 606/76

(57) ABSTRACT

A pre-configured implant cover fabricated from a drug-eluting biocompatible matrix containing at least one elutable drug, a kit containing at least one elutable drug implant cover, and a method of using the same.

### BACKGROUND OF THE INVENTION

This invention relates generally to drug-eluting or drug-diffusing cover for implants. More particularly, the invention is for a drug-eluting cover for orthopedic implant having such capability.

Numerous orthopedic implants, including spinal implants such as anterior spinal plates, are known to possess adherent coatings, layers or films containing one or more drugs, e.g., medicaments, therapeutics, biologicals or other bioactive substances, etc., such as antimicrobials, antibacterials, antibiotics, antifungicides, anti-inflammatories, and the like. Following the installation of such an implant in the body, the drug(s) present in the coating elutes therefrom over time into the region of surrounding tissue to achieve the desired drug actions(s).

However, these coatings, layers or films containing one or more drugs are in the form of a non-removable coating that is applied to the implant at the time of manufacture under special manufacturing conditions. One problem that is encountered in the manufacture of implants possessing a drug-eluting coating involves the sterilization of such a device. The more economical methods of sterilization utilize steam under pressure, e.g., as produced in an autoclave. While such sterilization methods are known to be highly effective, they are subject to a major disadvantage where thermally sensitive drugs are concerned and therefore are of limited use. While the conventional use of sterilizing radiation or a sterilant gas such as ethylene oxide can reduce the risk of damaging or partially to completely inactivating the drug component(s) present in the coating component of an orthopedic implant, such sterilization methods are relatively expensive. While it is possible in principle to apply a drug-containing coating to a pre-sterilized implant under sterile conditions followed by the sterile packaging of the coated implant, such an approach to providing a packaged sterile orthopedic implant which avoids subjecting the drug(s) contained in its drug-eluting coating to thermal decomposition or deactivation is largely an impractical one.

In addition, once the coating containing comprising the eluting drug is applied to the implant the eluting drug cannot be changed by the surgeon according to the particular conditions at the time of surgery. In order to provide the surgeon with different concentrations and/or drug compositions in the coating of an implant, several different pre-coated implants must be available in the operating room at the time of surgery and often these implants must be prepped for implant. Part of the prepping procedure is to remove the implant from the sterile enclosure in order to wash it with physiogical solution and have it ready for the surgeon upon request. Once removed from the package, whether used or not, the implant would have to be resterilized or discarded. Since, as stated above, the coating is already on the implant it is difficult to sterilize the unused implants without assuring that the eluting drug is still effective. For these reasons, providing several implants having different drugs/concentrations is often not done since the cost of discarding the implants is prohibitive.

Therefore, what is needed is a drug-eluting cover that can be applied to an implant in vivo or in vitro that can be sterilized separately from the implant and configured to fit a variety of implants is needed. The present invention provides such device and is described in further detail below.

### SUMMARY OF THE INVENTION

In accordance with the present invention, there is provided a drug-eluting implant cover comprising a pre-configured implant cover fabricated from a drug-eluting biocompatible matrix containing at least one elutable drug. In one embodiment of the present invention, the pre-configured implant is molded using standard mold technology so as to produce a pre-molded implant cover fabricated from a drug-eluting biocompatible matrix containing at least one elutable drug. The pre-molded implant can be made to fit a specific implant or a range of different implants. The drug-eluting implant cover of the present invention maybe made from an elastomeric material having a built-in memory so that when the elastomeric material is expanded and positioned to fit around at least a portion of an implant and released so as to contour to the shape of at least a portion of the implant so as to cover at least a portion of implant.

In accordance with the present invention, there is provided a method for covering an implant with a drug eluting material comprising providing an implant of the present invention and at least one drug-eluting implant cover of the present invention; and affixing the drug-eluting implant cover to at least a portion of the implant to produce an implant having a drug-eluting implant cover on at least a portion thereon. According to another aspect of the invention a method is provided using more than one cover can be placed on the implant having the same of different drug or concentration of the drug. This allows a wide variety of possibilities for the surgeon in the operation. The present also does not exclude the possibility of placing the cover on an implant already having a coating affixed thereto to provide additional drug eluting capabilities.

According to a further aspect of the invention, there is provided a method for affixing the drug eluting cover of the present invention to at least a portion of an implant prior to, during or following the installation of the implant into a patient.

According to yet another aspect of the invention, there is provided a drug-eluting implant cover kit comprising at least one implant cover fabricated from a drug-eluting biocompatible matrix containing at least one elutable drug in a sterile container. Kits that fall within the scope of the present invention can also include multiple implant covers having the same or different sizes, having the same or different drugs impregnated in the covers and/or having the same or different concentrations of the elutable drugs impregnated in the cover, all or part of which may be in a sterile container.

For the purpose of this application the following definitions are provided to aid in the understanding of the invention.

The term “implant” shall be understood herein to include, orthopedic implants, spinal implants, spinal stabilization implants, spinal dynamic implants, spinal rods, spinal plates, spinal interbody fusion devices, bone screws, pedicle screws, crosslink components, spinal hooks, interspinous process spacers, etc.

The term “preformed” as it is applied to the drug-eluting device component of the invention is to be understood as distinguishing the drug-eluting device from a drug-eluting coating that is manufactured upon a surface of an implant, such as an orthopedic implant. Thus, the drug-eluting cover that can be affixed to at least a portion of the implant, in contrast to known orthopedic implants possessing a drug-eluting coating, film, or layer manufactured thereon, is not produced upon a surface of the implant but upon some other surface if, indeed, it is produced upon a surface of any substrate at all, and detached so that the cover can be applied to the surface of an implant. That is, it is only after the fabrication of the drug-eluting cover that the cover of the present invention is affixed to the implant.

The term “biocompatible” as applied to the drug-eluting material from which the drug-eluting device herein is fabricated shall be understood in its ordinary art-recognized sense as describing a material exhibiting a relatively low chronic tissue response for the period that the material is present in the body.

The expression “drug-eluting” shall be understood to refer to any and all mechanisms, e.g., diffusion, migration, permeation, and/or desorption by which the drug(s) incorporated in the matrix pass therefrom over time into the surrounding body tissue.

The expression “drug-eluting matrix” shall be understood herein to mean any natural, synthetic or semi-synthetic material capable of acquiring and retaining a desired shape or configuration and into which one or more drugs can be incorporated and from which the incorporated drug(s) are capable of eluting over time.

The expression “elutable drug” shall be understood to mean a drug having the ability to pass over time from the drug-eluting matrix in which it is incorporated into the surrounding areas of the body.

The term “drug” includes all medically useful bio-affecting and body-treating compositions.

Other than where expressly indicated, all numbers expressing amounts of materials, concentrations, quantified properties of materials, and so forth, stated in the specification and claims are to be understood as being modified in all instances by the term “about” or “approximately.”

It will also be understood that any numerical range recited herein is intended to include all sub-ranges within that range and any combination of the various endpoints of such ranges or subranges.

It will be further understood that any compound, material or substance which is expressly or implicitly disclosed in the specification and/or recited in a claim as belonging to a group of structurally, compositionally and/or functionally related compounds, materials or substances includes individual representatives of the group and all combinations thereof.

The distinction between a drug-eluting coating as utilized by heretofore known orthopedic implants and the preformed drug-eluting cover of this invention is a fundamental one and is of considerable significance for addressing the sterilization problem discussed above. Thus, the implant and the drug-eluting structure of this invention can be supplied to the orthopedic surgeon as two separately sterilized components, one being the implant which has been sterilized by the economical autoclave method and the other being a preformed drug-eluting cover which has been sterilized by some other method, e.g., the use of sterilizing radiation or sterilant gas, that does not subject the drug(s) present therein to any significant level of decomposition, denaturation or deactivation. The surgeon then has the choice of affixing the drug-eluting cover to the implant just prior to, during or just after installation of the implant in the body as the particular circumstances may require.

Another major advantage of the implant of the present invention is that it can be assembled at the time of installation from a specific implant and a specific drug-eluting cover which can be selected from amongst a variety of such devices, each differing in the nature and/or amounts of the drug(s) contained therein and/or the nature of the drug-eluting composition, or matrix, from which the device is fabricated thereby offering the surgeon considerable flexibility for choosing the optimal implant and the optimal preformed drug-eluting cover or multiple covers for a particular patient's circumstances and needs. It is far more practical to provide such flexibility of choice in the case of an in situ assembled drug-eluting cover as in the present invention than to provide the same number of choices for a pre-coated implant of the prior art. To illustrate this, consider the case where a surgeon desires to choose from among 5 different sizes, designs or configurations of implants and five different varieties of drug-eluting material. In the case of the in situ assembled cover of the present invention, the surgeon need only have on hand 5 choices of implants and 5 choices of pre-formed drug-eluting covers (for a total of just 10 pre-assembly units) to meet all contemplated situations. However, it would require at least 25 pre-coated implants plates of the prior art to provide the same total number of choices. Once the sterility of the pre-coated implants has been compromised in the operating room, the unused pre-coated implants would need to be re-sterilized which, as stated above, requires special equipment and may compromise the drug within the drug eluting coating. Therefore, this is often not done and the surgeon is not given these choices.

Since the drug eluting cover can be packaged separately in sterile packages and the uncoated implants can be easily sterilized using ordinary autoclave machinery, the cost to provide these choices to the surgeon becomes economically feasible when using the present invention. In other words, since an uncoated implant can easily be sterilized using common autoclave and the drug-eluting covers can be individually packaged, there will be little or no waste.

The foregoing scenario points to yet another advantage of the invention over the prior art, namely, it presents the surgeon with the opportunity to choose from among all suppliers' implants to which one or more preformed drug-eluting covers may or may not be affixed. The surgeon is therefore not limited to the specific pre-coated offerings of just one or a few suppliers but has as many choices in this regard as the then-current commercial market makes available.

### BRIEF DESCRIPTION OF THE DRAWINGS

FIG. 1 is a perspective view of one embodiment of an implant and a preformed drug-eluting cover of this invention about to be affixed to the implant;

FIG. 2 is a side elevation view of one embodiment of a cervical column having several drug-eluting covers affixed thereto;

FIG. 3 is a side elevation view of one embodiment of a cervical column having several drug-eluting covers affixed thereto;

FIG. 4 is a side elevation view of one embodiment of a cervical column having several drug-eluting covers affixed thereto;

FIG. 5 is a side elevation view of one embodiment of a cervical column having several drug-eluting covers affixed thereto;

FIG. 6 shows several side elevation views of cervical columns having different positioning of drug-eluting covers of the present invention affixed thereto;

FIG. 7 shows several side elevation views of cervical columns having different positioning of drug-eluting covers of the present invention affixed thereto and the drug zones that result;

FIG. 8 shows several side elevation views of cervical columns having drug-eluting covers of the present invention affixed at multi-level of the cervical column;

FIG. 9 shows several side elevation views of cervical columns having drug-eluting covers of the present invention affixed at multi-level of the cervical column;

FIG. 10 shows several side elevation views of cervical columns having drug-eluting covers of the present invention affixed at multi-level of the cervical column;

FIG. 11 shows a drug-eluting cover of the present invention in the form of a wrap;

FIG. 12 shows multiple wraps of the present invention installed on a rod installed on a rod attached to a cervical column;

FIG. 13 shows the sheath in the form of a roll; and

FIG. 14 shows a single wrap of the present invention installed on a rod attached to a cervical column.

### BRIEF DESCRIPTION OF THE INVENTION

Implants such as orthopedic prosthetic implant devices constructed of plastics, polymers, metals, ceramics or materials made from composites of these materials to address orthopedic injuries and deformities has become commonplace. Such implant devices typically have one or more surfaces that are placed in direct contact with living tissues and some devices include surfaces against which living tissues of the host slide or otherwise move in normal use. In this arena, concerns are sometimes raised about decreasing the invasiveness of the implants and the procedures for implanting them, improving implant integrity, and improving patient outcomes.

Despite the many positive benefits that are gained by the use of such implant devices, contact between the surfaces of the implant and soft tissues of the host, including muscle tissues, blood and the like, can produce unwanted results. For example, dynamic contact between the surfaces of the implant and soft tissue of the host can cause significant abrasive damage to fragile and sensitive human cells and tissues. These dynamic contacts can also cause a wide range of undesirable effects such as tissue and cell adhesion, irritation, inflammation, thrombogenicity (clotting of the blood), hemolysis, bacterial adhesion and infections, unwanted mineral deposits, and increased pain or limited motion, to name a few.

To over come many of these problems some implants can be coated with a drug eluting matrix that is chemically adhered to the surface of a specific implant when manufactured and once applied to the implant can not be changed. For the reasons stated above, this is very limiting since the surgeon choices are limited. The present invention is directed to a drug-eluting cover that can be fitted onto an implant just prior to being implanted into a patient, thereby giving the surgeon maximum flexibility in choosing the particular drug, dosage or combination of drugs that is best for the patient. This drug eluting cover of the present invention is further described with the aid of the figures in the sections below.

For the purposes of promoting an understanding of the principles of the invention, reference will now be made to the embodiments illustrated herein and specific language will be used to describe the same. It will nevertheless be understood that no limitation of the scope of the invention is thereby intended. Any alterations and further modifications in the described processes, systems, or devices, and any further applications of the principles of the invention as described herein, are contemplated as would normally occur to one skilled in the art to which the invention relates.

Implants can be fabricated from a wide range of materials including metals, synthetic polymers, ceramics and bone. Examples of these materials include metals such as medical grade stainless steel, titanium and titanium alloys, and the like, synthetic polymers such as thermoplastic polymers, thermoset polymers, elastomers, and the like, ceramics such as pyrolytic carbon, carbon fibers, and their composites, zirconia, alumina, titanic and their composites, and the like, bone, e.g., autograft, allograft, xenograft or transgenic cortical and/or corticocancellous bone obtained, e.g., from the femur, tibia, fibula radius and/or ulna and provided as a single unit or as a composite built up from smaller bone elements and/or bone-derived particles. Examples of non-resorbable polymers include silicone, polyurethane, silicone-polyurethane copolymers, polyethylene, polypropylene, polyester, polyaryletherketone, polyimide, polyetherimide, polyamide, polysulfone. Examples of resorbable polymer include polylactide, polyglycolide, copolymers of polylactide and polyglycolide, polycaprolactone, and polyorthoester.

These implants come in many different sizes and configurations for installation at various cervical, thoracic and lumbar regions of the spine. This implants can be held in place by a variety of fastener components such as screws, rods and rod connectors. Some implants can be provided as an assemblage of two or more sub-units in which these fastener components can be used to connect. Illustrative spinal plates are those described in U.S. Pat. Nos. 6,193,721; 6,206,882; 6,224,602; 6,228,085; 6,258,089; 6,342,055; 6,413,259; 6,533,786; 6,602,255; 6,602,256; 6,605,090; 6,613,053; 6,679,883; 6,755,833; 6,761,719; 7,041,105; 7,169,150; 7,186,256; 7,306,605; 7,468,069; and, 7,481,829, and U.S. patent application publications 2004/0204712; 2005/0192577; 2005/0228386; 2007/0043369; 2007/0233110; 2007/0276371; 2008/0234753; 2009/0012571; and, 2009/0024171, the entire contents of which are incorporated by reference herein.

Coating containing drug reservoirs that are distinguishable from the present invention have been described in U.S. Patent Nos. 2005/0031666; 2007/0299520; 2006/0047341; 2007/0270858; 2004/0030342; 2007/0173934, the entire contents of which are incorporated by reference herein.

Prior to, during or following the surgical installation of a selected implant, the drug eluting cover of the present invention can be stretched and positioned over the portion of the implant in which the surgeon believes is most beneficial. Once in position the stretched cover attempts to return back to its shape and contours to the shape of the implant. In most cases this tension or clinging to the surface of the implant may be enough to hold the cover implant or in the alternative additional affixing devices may be required. For example, a biologically acceptable adhesive can be used to hold the drug-eluting cover in place.

The drug-eluting matrix can possess a planar shape, e.g., that of a square, rectangle, circle, oval, etc., which can be wrapped around at least a portion of the implant and affixed in place. In the alternative, the drug eluting cover can be in the form of a sleeve that can be slipped over the implant and held in place by the natural clinging of the elastic cover to at least a portion of the implant as described above, or can be affixed by other means including a biological adhesive. Still further the drug eluting cover can be produced in sheets or long sleeves that can be cut to size by the surgeon at the time of surgery. This giving the maximum choices to the surgeon.

The matrix can be formed from a material of homogeneous or heterogeneous composition, can possess a single layer or multiple layers (i.e., a laminate), can be rigid, flexible or semiflexible, can be stretchable (elastic) so as to engagedly fit some portion of its associated implant or nonstretchable (inelastic), can be porous or non-porous, can vary considerably in its average dimensions, etc. The drug eluting cover can have at least one extension configured to fit within a complimentary cavity located on the surgical implant. The drug eluting cover can be positioned so that the extension (or extensions) snap (or pressure fit) into the surgical implant. In this embodiment, the surface of the drug-eluting matrix is in direct contact with at least one surface of the implant.

Since no contact between surfaces is absolutely perfect, gaps and/or air pockets can and will arise between the drug eluting matrix and the surface of the implant. These gaps and/or pockets can develop infections which can be prevented by use of antimicrobial/anti-infectious compounds which elude from the matrix into the pockets that may form. In addition to preventing infection in these gap/pockets, the drug-eluting matrix also eludes into the soft tissue surrounding the implant. The present invention is configured to allow the drug to elute from the matrix towards the surface of the implant as well as to the surrounding soft tissue. This feature aids in preventing any infections that may arise between the drug eluting layer and the surface of the implant as well in the surrounding tissue.

The drug-eluting cover can be dimensioned and configured as desired by any suitable technique, e.g., molding, machining, die-cutting from a larger sheet or section, etc., and can be dimensioned and configured by the surgeon or assistant personnel, e.g., by scissors if the nature of the drug-eluting matrix permits, at or near the time the drug-eluting cover is to be used affixed to the selected implant.

The drug-eluting matrix component of the drug-eluting device can be fabricated from amongst any of the numerous biocompatible materials heretofore known for providing drug-eluting devices. Useful matrices include non-bioresorbable, or non-bioabsorbable, materials and bioresorbable, or bioabsorble, materials. Natural, semi-synthetic and fully synthetic polymers of both types are well known in the art for use as drug-eluting coatings.

Among the useful non-bioresorbable drug-eluting matrices are polyurethanes, silicones (polysiloxanes), polyesters, polyamides, polyolefins such as polyethylene, polypropylene, polyisobutylene and their copolymers, acrylic polymers and copolymers, vinyl halide polymers and copolymers, polyvinyl ethers, polyacrylonitriles, polyvinyl ketones, polyvinyl aromatics such as polystyrene, polyvinyl esters, polycarbonates, polyimides, polyethers, epoxy resins, and the like.

Useful bioresorbable drug-eluting matrices include hydrogels, and polymers such as poly(L-lactic acid), poly(glycolic acid), poly(lactide-co-glycolide), polydioxanone, polyorthoesters, polyanhydrides, and the like. For example, such polymers include but are not limited to a polyvinyl alcohol, a polyacrylic acid, a polyarylamide, a poly(acrylonitrile-acrylic acid), a polyurethane, a polyethylene glycol, a poly(N-vinyl-2-pyrrolidone), a gelatin, a collagen, a polysaccharide, a cellulose, and combinations thereof. The hyrdogels of the present invention may be hydrated or unhydrated. The unhydrated hydrogels of the present invention will become hydrated either prior, during or after implantation. Once hydrated the hydrogels will increase in dimensions. The present invention can be made from hydrogels either impregnated with and/or coated with antibacterial/antimicrobial agents such as silver atoms, silver ions and/or mixtures thereof.

Hydrogels that can be used for the present invention may also be made non-bioresorbable by means of the process in which they are produced as well as the molecular composition. Various degrees of bioresorbability can also be accomplished by varying the amount of crosslinking in the hydrogel.

If desired, the drug-eluting device herein can be provided as a laminate with, e.g., a first layer (the layer closest to the surface of the anterior spinal plate to which the device will be affixed) fabricated from a non-bioresorbable matrix containing one or more elutable drugs and superimposed thereon a second layer of bioresorbable matrix containing the same or different drug(s) as the first layer.

The drug-eluting properties of a drug-eluting matrix, principally the rate of release of its drug component(s) into the surrounding body tissues, is of prime importance. Those skilled in the art employing known procedures can readily select the optimum drug-eluting matrix material for a particular drug or drug combination and drug loading(s). The selected drug(s) can be incorporated in the drug-eluting matrix during and/or after the formation of the matrix material of the cover. The incorporation of drug can be substantially uniform or the drug(s) can be distributed in the matrix in gradient fashion or in distinct zones of concentration employing any of several methods known in the art. Thus, e.g., a greater concentration of drug(s) at or near the exposed surface of the matrix can be made to provide an initial higher concentration of drug(s) in the surrounding tissues followed by a reduction in delivered drug concentration (and perhaps longer term drug delivery as well if desired) as the more interior regions or zones of lower drug concentration within the drug(s). This gradient or zonal distribution of drug in the drug-eluting matrix can be utilized to initially deliver a higher concentration of one drug in a drug combination followed by later delivery of a higher concentration of another drug in the drug combination.

Useful drug incorporation procedures include combining the selected elutable drug(s) with the precursor(s) of the matrix and thereafter forming the cover. Thus, in the case of a polymeric matrix, e.g., an open cell polyurethane foam, the drug(s) can be admixed with the precursor reactants (e.g., polyisocyanate and polyol among other components of the polyurethane foam-forming reaction mixture) with the resulting polyurethane foam entraining the drug(s).

Another drug incorporation procedure involves contacting the drug-eluting matrix material with a drug-containing solvent medium which dissolves the matrix and following evaporation of the solvent(s) leaves the drug(s)in the reconstituted matrix. A similar procedure involves contacting the matrix with a drug containing swelling agent and allowing the drug(s) to diffuse into the matrix of the cover.

When an open cell matrix is used as the drug-eluting vehicle, e.g., the aforementioned polyurethane foam, the desired drug(s) can be incorporated in the matrix by immersion in a suitable aqueous and/or organic solvent solution of the drug(s) followed by draining excess solvent and if desired, drying.

The drug-eluting matrix can also be fashioned from organic and/or inorganic particulate material and drug bonded together in the desired configuration employing a biocompatible bonding or binder material. Examples of a binder material include the resorbable or non-resorbable biomaterials mentioned above. Additional examples of a binder material include those used in pharmaceutical industry such as a polysaccharide material, a cellulose material, a collagen material, a gelatin material, a synthetic bioresorbable polymer, etc.

These and/or other known techniques can also be used to incorporate one or more non-drug materials in the matrix component of the drug-eluting device herein. Among some optional non-drug materials that can be incorporated in the drug-eluting matrix are diluents, carriers, excipients, stabilizers, permeation enhancers, surface active agents, and the like, in known and conventional amounts.

The amounts of elutable drug for incorporation in the drug-eluting matrix herein will depend on a number of factors well understood by those skilled in the art including the nature of the selected drug(s), the nature, amounts and configuration of the selected matrix and the desired profile (rate and duration) of drug release into the surrounding tissues. Again, empirical investigation employing known and conventional procedures can be utilized by those skilled in the art to arrive at an optimum concentration of specific drug(s) for a specific matrix arrangement. The concentration of drug(s) and the drug-eluting profile of the matrix component of the drug-eluting device will be such as to deliver a therapeutically effective concentration of the desired drug(s) for a therapeutically useful duration. Total concentration of deliverable drug can range, e.g., from about 0.1% to about 20%, and preferably 1% to about 10%, weight percent of the drug-eluting matrix and can provide eluted drug(s) in therapeutically useful amounts for periods ranging e.g., for at least 24 hours and preferably at least 70, 100, 250, 500 or even 750 hours or more. In certain embodiments, the duration of effective drug release can be range from 1 to 6 months.

As previously indicated, the dimensions of the drug-eluting cover can vary considerably. Thus, the surface dimensions of the cover can be configured so as to exceed, match or be less than that of the surface of the implant or implants to which it can be used. By way of illustration, in the case of a spinal implant having an average major surface dimension of about 100 mm and a minor surface dimension of about 6 mm, the drug-eluting cover can possess a length of from about 5 mm to about 100 mm and a width of from about 1 mm to about 6 mm.

The thickness of the drug-eluting cover can influence not only the rate of drug release from the device but also the elasticity of the cover which is one way in which the cover of the present invention is held in place. Although these dimensions can vary considerably depending on the drug release profile desired, one should also consider the elasticity of the cover once formed. In one embodiment, the thickness of the drug-eluting cover ranges, e.g., from about 0.1 mm to about 5 mm and preferably from about 0.5 mm to about 2 mm.

The drug(s) selected for incorporation in the drug-eluting cover can be essentially pure and/or concentrated and can be in the form of a solid e.g., a powder, a semi-solid, e.g. a gel, a paste, a slurry or a liquid, e.g. a solution, or a suspension of widely varying appearance. The physical properties and characteristic elution rates from a given drug-eluting cover can be determined by a person of ordinary skill in the art, including when the drug is encased in a dissolvable solid bead or liposome for delayed release of the drug. When desired, a drug can be incorporated in the cover in both an encapsulated form and/or a free form via suitable carrier liquids, e.g., solvents, in particular, water, organic solvent(s) or aqueous mixtures of organic solvent(s). In addition, the cover may optionally contain one or more non-drug materials, e.g., one or more of those previously recited components, dissolved, suspended or dispersed therein. It will, of course, be appreciated that when the physical form of the pure and/or concentrated drug is that of a solid or semi-solid, it may be beneficial if at least some portion of the carrier with the drug(s) dissolved, suspended or dispersed therein is retained in the polymer matrix for subsequent delivery of such drug(s) to the surrounding region of tissue.

The drug, or drugs, incorporated in the drug-eluting cover herein include, but are not limited to, anti-infective agents such as antibiotics, antiseptics, antiviral agents and anti-fungal agents, anti-inflammatory agents, local anesthetics and/or any of numerous other classes of therapeutic agents.

Any antibiotic suitable for use in a human may be used in accordance with various embodiments of the invention. As used herein, “antibiotic” means an antibacterial agent. The antibacterial agent may have bateriostatic and/or bacteriocidal activities. Nonlimiting examples of classes of antibiotics that may be used include tetracyclines (e.g. minocycline), rifamycins (e.g. rifampin), macrolides (e.g. erythromycin), penicillins (e.g. nafcillin), cephalosporins (e.g. cefazolin), other beta-lactam antibiotics (e.g. imipenem, aztreonam), aminoglycosides (e.g. gentamicin), chloramphenicol, sufonamides (e.g. sulfamethoxazole), glycopeptides (e.g. vancomycin), quinolones (e.g. ciprofloxacin), fusidic acid, trimethoprim, metronidazole, clindamycin, mupirocin, polyenes (e.g. amphotericin B), azoles (e.g. fluconazole) and beta-lactam inhibitors (e.g. sulbactam). Nonlimiting examples of specific antibiotics that may be used include minocycline, rifampin, erythromycin, nafcillin, cefazolin, imipenem, aztreonam, gentamicin, sulfamethoxazole, vancomycin, ciprofloxacin, trimethoprim, metronidazole, clindamycin, teicoplanin, mupirocin, azithromycin, clarithromycin, ofloxacin, lomefloxacin, norfloxacin, nalidixic acid, sparfloxacin, pefloxacin, amifloxacin, enoxacin, fleroxacin, temafloxacin, tosufloxacin, clinafloxacin, sulbactam, clavulanic acid, amphotericin B, fluconazole, itraconazole, ketoconazole, and nystatin. Other examples of antibiotics, such as those listed in U.S. Pat. No. 4,642,104, the entire contents of which are incorporated by reference herein, may also be used. One of ordinary skill in the art will recognize other antibiotics that may be used.

In general, it is desirable that the selected antibiotic(s) kill or inhibit the growth of one or more bacteria that are associated with infection following surgical implantation of a medical device. Such bacteria are recognized by those of ordinary skill in the art and include Staphylococcus aureus, Staphylococcus epidermis, and Escherichia coli. Preferably, the antibiotic(s) selected are effective against strains of bacteria that are resistant to one or more antibiotic.

To enhance the likelihood that bacteria will be killed or inhibited, it may be desirable to combine two or more antibiotics. It may also be desirable to combine one or more antibiotic with one or more antiseptic. It will be recognized by one of ordinary skill in the art that using two or more antimicrobial agents having different mechanisms of action and/or different spectrums of action may be most effective in achieving the desired effect. In one embodiment, a combination of rifampin and minocycline is used, e.g., at a rifampin loading of from about 0.1% to about 20%, preferably from about 1% to about 10%, and a minocyline loading of from about 0.1% to about 20% and preferably from about 1% to about 10%. In another embodiment, rifampin at one of the aforementioned loadings can be combined with clindamycin at a loading of from about 0.1% to about 20% and preferably from about 1% to about 10%.

Any antiseptic suitable for use in a human may be used in accordance with various embodiments of the invention. As used herein, “antiseptic” means an agent capable of killing or inhibiting the growth of one or more of bacteria, fungi, or viruses. Antiseptic includes disinfectants. Nonlimiting examples of antiseptics include hexachlorophene, cationic bisiguanides (i.e. chlorhexidine, cyclohexidine) iodine and iodophores (i.e. povidone-iodine), para-chloro-meta-xylenol, triclosan, furan medical preparations (i.e. nitrofurantoin, nitrofurazone), methenamine, aldehydes (glutaraldehyde, formaldehyde), silver-containing compounds (silver sulfadiazene, silver metal, silver ion, silver nitrate, silver acetate, silver protein, silver lactate, silver picrate, silver sulfate), and alcohols. One of ordinary skill in the art will recognize other antiseptics that may be employed in accordance with this disclosure.

It is desirable that the antiseptic(s) selected kill or inhibit the growth of one or more microbe that are associated with infection following surgical implantation of a medical device. Such microbes are recognized by those of ordinary skill in the art and include Staphylococcus aureus, Staphylococcus epidermis, Escherichia coli, Pseudomonas aeruginosa, and Candidia.

To enhance the likelihood that microbes will be killed or inhibited, it may be desirable to combine two or more antiseptics. It may also be desirable to combine one or more antiseptics with one or more antibiotics. It will be recognized by one of ordinary skill in the art that antimicrobial agents having different mechanisms of action and/or different spectrums of action may be most effective in achieving such an effect. In a particular embodiment, a combination of chlorohexidine and silver sulfadiazine is used.

Any antiviral agent suitable for use in a human may be used in accordance with various embodiments of the invention. Nonlimiting examples of antiviral agents include acyclovir and acyclovir prodrugs, famcyclovir, zidovudine, didanosine, stavudine, lamivudine, zalcitabine, saquinavir, indinavir, ritonavir, n-docosanol, tromantadine and idoxuridine. One of ordinary skill in the art will recognize other antiviral agent that may be employed in accordance with this disclosure.

To enhance the likelihood that viruses will be killed or inhibited, it may be desirable to combine two or more antiviral agents. It may also be desirable to combine one or more antiseptics with one or more antiviral agent.

Any anti-fungal agent suitable for use in a human may be used in accordance with various embodiments of the invention. Nonlimiting examples of anti-fungal agents include amorolfine, isoconazole, clotrimazole, econazole, miconazole, nystatin, terbinafine, bifonazole, amphotericin, griseofulvin, ketoconazole, fluconazole and flucytosine, salicylic acid, fezatione, ticlatone, tolnaftate, triacetin, zinc, pyrithione and sodium pyrithione. One of ordinary skill in the art will recognize other anti-fungal agents that may be employed in accordance with this disclosure.

Any anti-inflammatory agent suitable for use in a human may be used in accordance with various embodiments of the invention. Non-limiting examples of anti-inflammatory agents include steroids, such as cortisone, hydrocortisone, prednisone, dexamethasone, methyl-prednisilone, an, derivatives thereof; and non-steroidal anti-inflammatory agents (NSAIDs). Non-limiting examples of NSAIDS include ibuprofen, flurbiprofen, ketoprofen, aclofenac, diclofenac, aloxiprin, aproxen, aspirin, diflunisal, fenoprofen, indomethacin, mefenamic acid, naproxen, phenylbutazone, piroxicam, salicylamide, salicylic acid, sulindac, desoxysulindac, tenoxicam, tramadol, ketoralac, flufenisal, salsalate, triethanolamine salicylate, aminopyrine, antipyrine, oxyphenbutazone, apazone, cintazone, flufenamic acid, clonixerl, clonixin, meclofenamic acid, flunixin, coichicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, and triflumidate.

Any local anesthetic agent suitable for use in a human may be used in accordance with various embodiments of the invention. Non-limiting examples of local anesthetics agents include lidocaine, prilocaine, mepivicaine, benzocaine, bupivicaine, amethocaine, lignocaine, cocaine, cinchocaine, dibucaine, etidocaine, procaine, veratridine (selective c-fiber blocker) and articaine.

Non-limiting examples of other pharmacological agents that may be used include: beta-radiation emitting isotopes, beclomethasone, fluorometholone, tranilast, ketoprofen, curcumin, cyclosporin A, deoxyspergualin, FK506, sulindac, myriocin, 2-aminochromone (U-86983), colchincines, pentosan, antisense oligonucleotides, mycophenolic acid, etoposide, actinomycin D, camptothecin, carmustine, methotrexate, adriamycin, mitomycin, cis-platinum, mitosis inhibitors, vinca alkaloids, tissue growth factor inhibitors, platinum compounds, cytotoxic inhibitors, alkylating agents, antimetabolite agents, tacrolimus, azathioprine, recombinant or monoclonal antibodies to interleukins, T-cells, B-cells, and receptors, bisantrene, retinoic acid, tamoxifen, compounds containing silver, doxorubicin, azacytidine, homoharringtonine, selenium compounds, superoxide-dismutase, interferons, heparin; antineoplastic/antiangiogenic agents, such as antimetabolite agents, alkylating agents, cytotoxic antibiotics, vinca alkaloids, mitosis inhibitors, platinum compounds, tissue growth factor inhibitors, cisplatin and etoposide; immunosuppressant agents, such as cyclosporine A, mycophenolic acid, tacrolimus, rapamycin, rapamycin analogue (ABT-578) produced by Abbott Laboratories, azathioprine, recombinant or monoclonal antibodies to interleukins, T-cells, B-cells and/or their receptors; anticoagulents, such as heparin and chondroiten sulfate; platelet inhibitors such as ticlopidine; vasodilators such as cyclandelate, isoxsuprine, papaverine, dipyrimadole, isosorbide dinitrate, phentolamine, nicotinyl alcohol, co-dergocrine, nicotinic acid, glycerl trinitrate, pentaerythritol tetranitrate and xanthinol; thrombolytic agents, such as stretokinase, urokinase and tissue plasminogin activators; analgesics and antipyretics, such as the opioid analgesics such as buprenorphine, dextromoramide, dextropropoxyphene, fentanyl, alfentanil, sufentanil, hydromorphone, methadone, morphine, oxycodone, papaveretum, pentazocine, pethidine, phenopefidine, codeine dihydrocodeine; acetylsalicylic acid (aspirin), paracetamol, and phenazone; and, antiproliferative agents such as QP-2 (taxol), paclitaxel, rapamycin, tacrolimus, everolimus, actinomycin, methotrexate, angiopeptin, vincristine, mitocycin, statins, C-MYC antisense, sirolimus, restenASE, 2-chloro-deoxyadenosine, PCNA (proliferating cell nuclear antigent) ribozyme, batimastat, prolyl hydroxylase inhibitors, halofuginone, C-proteinase inhibitors, and probucol; and combinations and/or derivatives thereof.

The following example is illustrative of the manufacture of the drug-eluting device of the anterior spinal plate of the invention.

### EXAMPLE

A sheath made of a silicone elastomer with a length of 10 mm, an inner diameter of 5.75 mm, and a thickness of 1 mm is used as a drug-eluting component for spinal rods. The sheath contains 4 weight percent of Rifampin and 6 weight percent of Minocycline. Alternately, the sheath contains 4 weight percent Rifampin and 6 weight percent Clindamycin. The drugs can be incorporated into the silicone before, during or after the curing of the silicone. In one example, the drugs can be mixed into a room temperature vulcanized silicone rubber prior to extrusion or molding. In an alternate example, the drugs may be incorporated via a solvent-swelling method. The sheath loaded with antimicrobial drugs is sterilized with ethylene oxide gas and supplied to the operating room in sterile packaging. The sheath is placed onto spinal rods that are 6 mm in diameter during surgery in order to provide the adjacent tissue areas with locally released antibiotics.

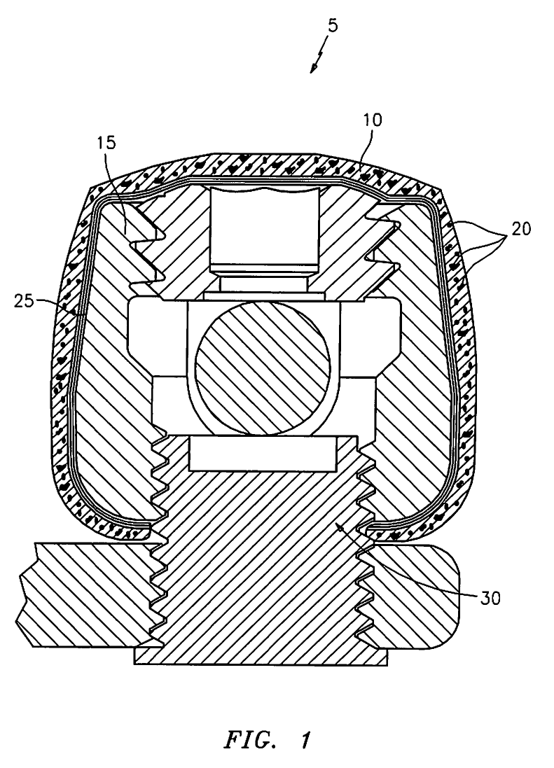

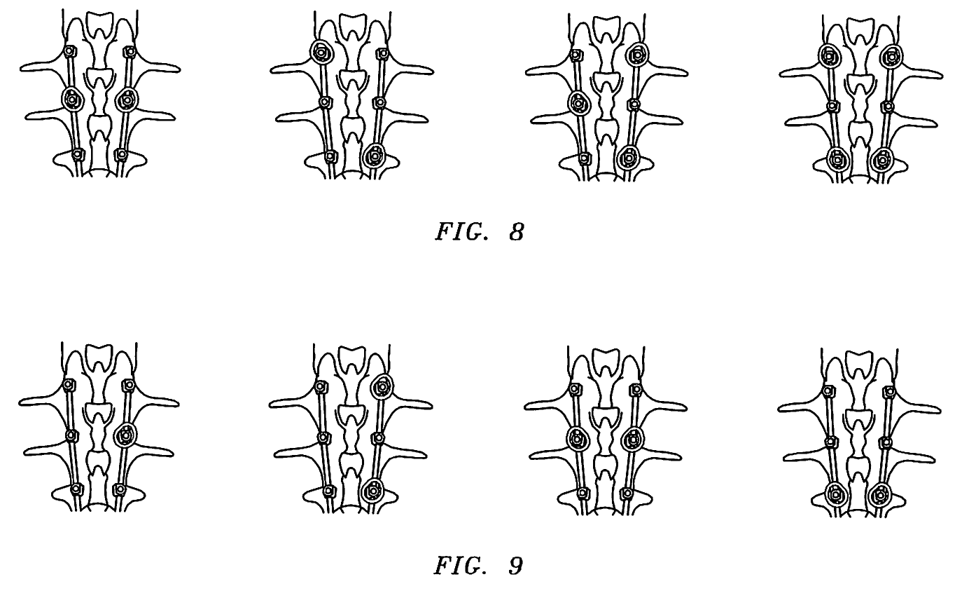

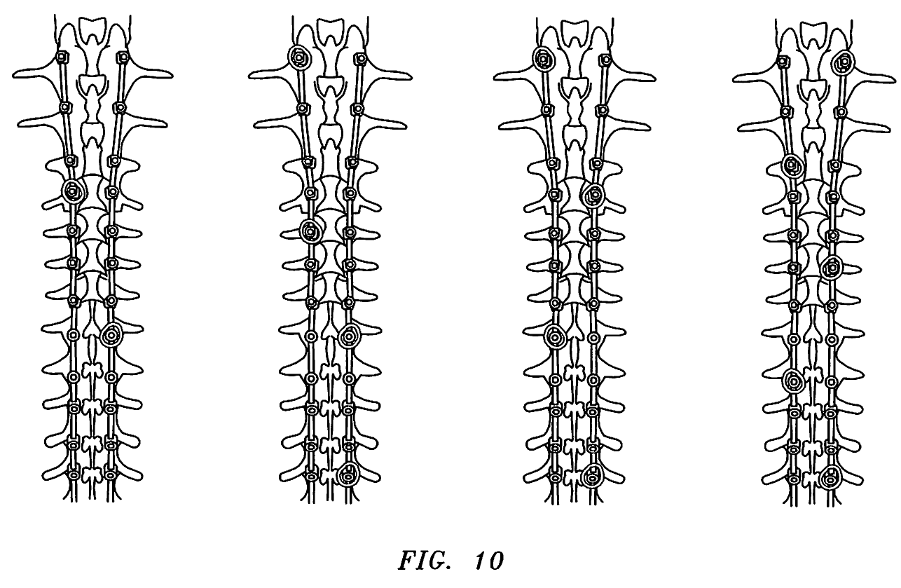

Specific embodiments and methods of using the same are described in conjunction with FIGS. 1-14. These figures are envisioned to help in describing the invention but are in no way meant to be limiting in scope of the invention.

FIG. 1 illustrates an implant having a drug eluting cover 05 already affixed thereto. Implant 15 can be attached to bone in the body by an attaching element 30 of the implant 15. A drug eluting cover of the present invention 10 can be placed over at least a portion of implant 15 so that one surface of the cover comes in contact with the surface 25 of the implant 15. As shown, the drug to be eluted from the cover 10 is indicated by the multiple of specks 20 dispersed throughout the cover 10. As stated the thickness of the cover and the concentration, and type of drug, can and will vary form cover to cover but are envisioned to fall within the present invention. Also as shown, although the cover is designed to make as close a contact as possible with the surface of the implant spaces and gaps do occur. Since the cover can elute antibacterial and the like either towards the soft tissues as well as towards the surface of the implant, infections can be kept to minimum.

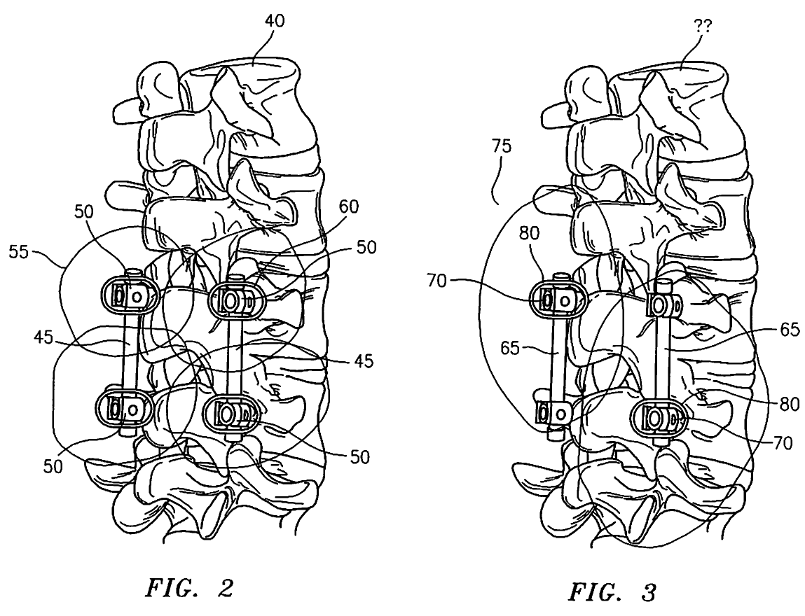

FIG. 2 illustrates a cervical column 40 having two rods 45 and four connectors 50. Each connector 50 is fitted with a drug eluting cover 60 of the present invention, which has a particular concentration of drug to be eluted to the surrounding areas. This figure demonstrates a situation where the concentration of drug in the drug eluting cover 60 of the present invention is low and therefore results in a relatively small range of drug elution creating a small drug area 55. Having a smaller range 55, more drug eluting covers 60 can be used so that the estimated drug eluting areas can overlap to provide a larger total drug zone. The number of cover used and the positioning of the covers can be determined by the surgeon either in advance or depending upon conditions observed at the time of surgery.

Accordingly, FIG. 3 illustrates a situation where the drug eluting covers 80 affixed on connectors 70 which are attach rods 65 to the cervical column 80 have a higher concentration than the covers shown in FIG. 2. For this reason, only two drug eluting covers 80 are used which produce larger drug eluting areas that overlap to provide an associated drug zone.

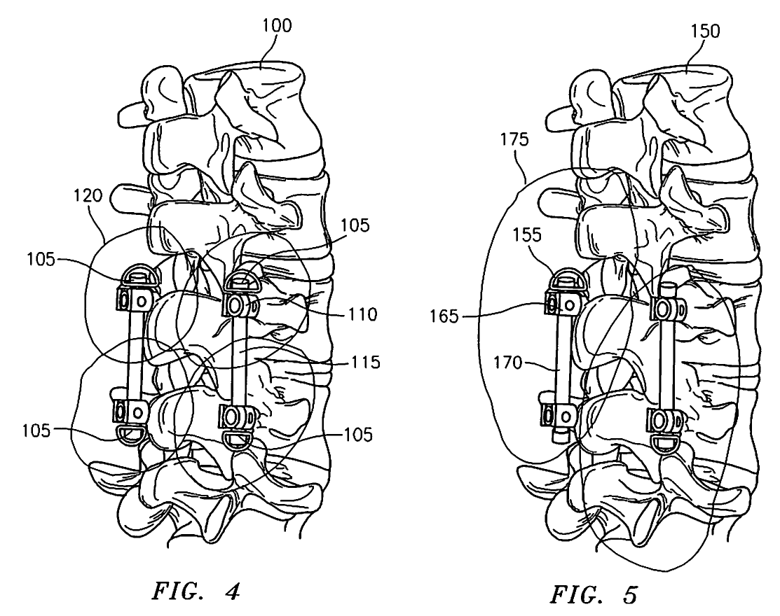

FIGS. 4 and 5 illustrate drug-eluting covers (120 in FIG. 4 and 155 in FIG. 5) affixed to the rod-ends of the rod and connector assemblies shown attached to a cervical column. In FIG. 4 the drug concentration of the drug-eluting covers is smaller than the concentration of the covers in FIG. 5 thereby producing a smaller drug eluting range than that of the covers shown in FIG. 5. Accordingly, fewer drug-eluting covers can be used in FIG. 5 than in FIG. 4.

The number of drug-eluting covers used in a particular implant surgery as well as the concentration of drug that each drug-eluting cover has can vary according to the particular needs. Each cover used can have the same or different drug concentrations thereby producing a variety of customized drug zones as may be necessary in each implant.

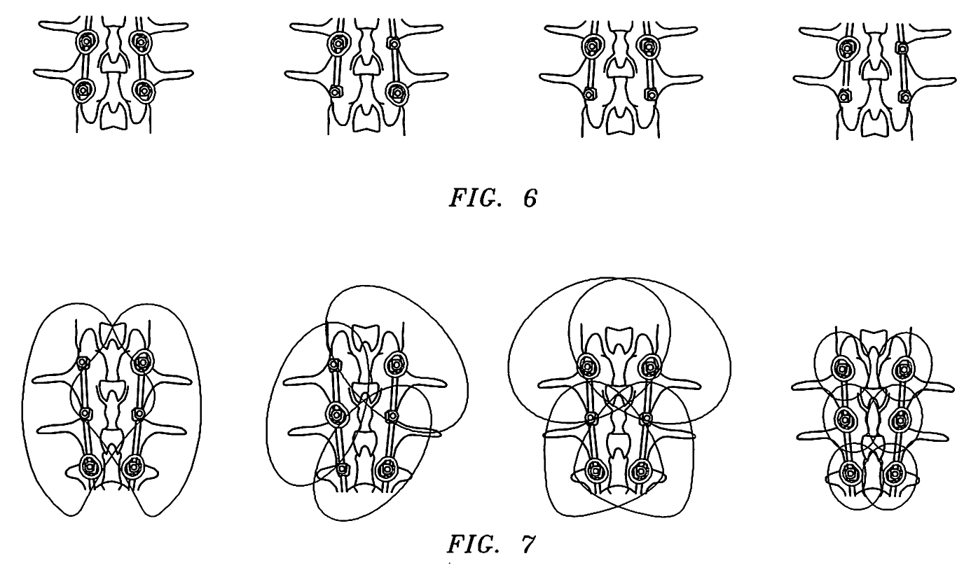

FIGS. 6 through 10 show different levels and/or combination of covers that can be used to provide customized drug delivery.

FIG. 11 shows a drug eluting cover in the form of a sheet that can be rolled onto itself to form a wrap that covers, for example rods used in spinal implants. These wraps can be held in place either by biological adhesives and/or securing staples, fasteners and the like can be used to affix the cover in the form of a sheath in place. In this configuration, the cover in the form of a sheet can be slid around portions of an implant and even an existing bone and secured in place to provide drugs to that localized area.

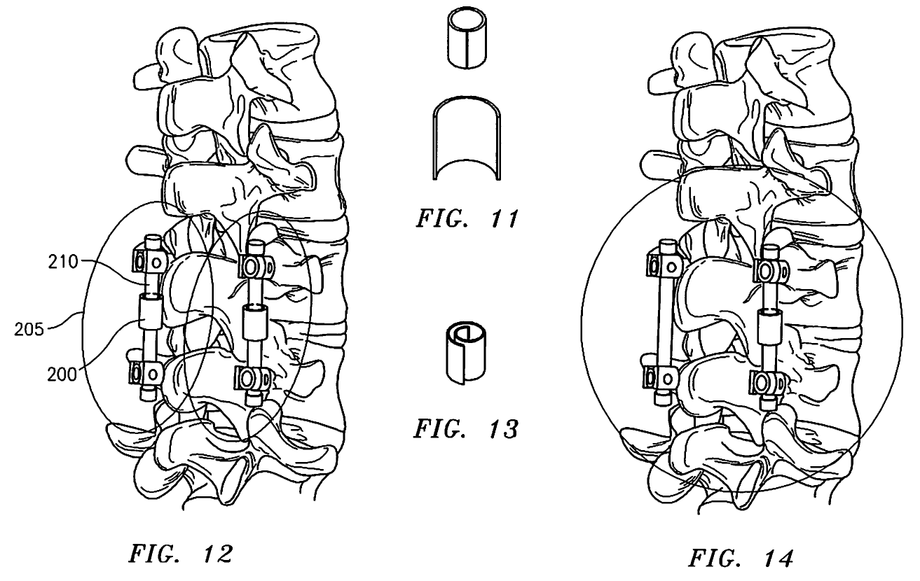

FIG. 13 shows the sheath of FIG. 11 in the form of a wrap. FIGS. 12 and 14 show the cover of the present invention 200 in the form of a sheath wrapped around a rod 210 already installed in a cervical column. As with the embodiments above, the concentration of drug to be eluted can vary so as to produce different drug zones as the drugs elute form the cover. The concentration, for the most part, is directly proportional to the drug zone produced around the cover. That is the higher the concentration, the larger the zone with the converse being true as well. FIG. 14 shows a higher concentration cover being used which produces a larger drug zone around it thereby requiring the installation of only one instead of two covers. Combinations of covers in the form of sheets, caps, and/or sheaths can be used to achieve the drug zones necessary for a successful outcome.

Other configurations may be possible that use the above-discussed structural and adhesive components to attach the drug-eluting matrix to the anterior spinal plate of the invention that are envisioned to fall within the inventive aspect of the invention.

While the invention has been illustrated and described in detail the drawings and foregoing description, the same is considered to be illustrative and not restrictive in character, it is understood that only the preferred embodiments have been shown and described and that all changes and modifications that come within the spirit of the invention are desired to be protected.

What is claimed is:

1. A drug-eluting implant cover comprising;
a pre-configured implant cover fabricated from a drug-eluting biocompatible matrix containing at least one elutable drug.

2. The drug-eluting implant cover of claim 1, wherein said implant cover is made from an elastomeric material having a built-in memory so that when said elastomeric material is expanded and released said elastomeric material contours to the shape of at least a portion of an implant so as to cover said portion of said implant.

3. The drug-eluting implant cover of claim 1, wherein said implant cover comprises a bioresorbable polymer.

4. The drug-eluting implant cover of claim 1, wherein said implant cover comprises a hydrogel selected from the group consisting of a polyvinyl alcohol, a polyacrylic acid, a polyarylamide, a poly(acrylonitrile-acrylic acid), a polyurethane, a polyethylene glycol, a poly(N-vinyl-2-pyrrolidone), a gelatin, a collagen, a polysaccharide, a cellulose, and combinations thereof.

5. The drug-eluting implant cover of claim 1, wherein the cover comprises a hydrogel having a water content when fully hydrated of at least 25% by weight.

6. The drug-eluting implant cover of claim 1, further comprising one or more tabs, flexible or elastic structures or locking elements to affix said implant cover to at least a portion of an implant.

7. The drug-eluting implant cover of claim 1, wherein the elutable drug is selected from the group consisting of at least one antimicrobial, antibacterial, antifungal, anti-inflammatory, analgesic, steroid, anti-adhesion agent, growth factor, wound-healing accelerator, immuno-suppressant, bone morphogenic protein and combinations thereof.

8. The drug-eluting implant cover of claim 1, wherein the elutable drug is monocycline, clindamycin, rifampin or a combination thereof.

9. The drug-eluting implant cover of claim 8, wherein said drug-eluting implant cover contains monocycline at a level of from about 0.5% to about 10% and/or clindamycin at a level of from about 0.5% to about 10% weight percent of a drug-eluting matrix in combination with rifampin at a level of from about 0.5% to about 10% weight percent of the drug-eluting matrix.

10. The drug-eluting implant cover of claim 1, wherein said drug-eluting matrix has a thickness of between about 0.1 mm and about 5 mm.

11. The drug-eluting implant cover of claim 1, wherein at least a portion of said implant cover is configured to cover at least a portion of an orthopedic connector.

12. The drug-eluting implant cover of claim 1, wherein at least a portion of said implant cover is flexible.

13. The drug-eluting implant cover of claim 1, which is in the form of a drug-eluting sheath.

14. The drug-eluting implant cover of claim 1, wherein the pre-configured implant cover is pre-molded.

15. The drug-eluting implant cover of claim 3, wherein the antibacterial/antimicrobial agent is selected from the group consisting of silver metal, silver ions, and mixtures thereof.

16. A drug-eluting implant cover kit comprising:
at least one implant cover fabricated from a drug-eluting biocompatible matrix containing at least one elutable drug in a sterile container.

17. The drug-eluting implant cover kit of claim 19, comprising multiple implant covers of the same or a different size in a sterile container, wherein at least two implant covers have drugs in different concentrations.

18. A method for covering an implant with a drug eluting material comprising:
a) providing an implant;
b) providing said drug-eluting implant cover of claim 1; and
c) affixing said drug-eluting implant cover to at least a portion of said implant to produce an implant having a drug-eluting implant cover on at least a portion of said implant.

19. The method of claim 18, wherein said affixing step is carried out prior to or during installing of said implant.

20. The method of claim 19, further comprising affixing the drug-eluting implant cover to the implant prior to installation using a biocompatible adhesive or stretching the drug-eluting implant cover to engagedly affix to a portion of said implant.

21. The method of claim 20, wherein said implant is an orthopedic rod-screw connector.
